(19) United States
(12) Patent Application Publication
(10) Pub. No.: US 2006/0281720 A1
(43) Pub. Date: Dec. 14, 2006

(54) 5-Androstenediol As An Inhibitor of Gliomas

(76) Inventor: Roger M. Loria, Richmond, VA (US)

Correspondence Address: CASTELLANO MALM FERRARIO & BUCK PLLC, 1250 CONNECTICUT AVENUE NW, SUITE 200, WASHINGTON, DC, 20036, US

(21) Appl. No.: 11/422,820

(22) Filed: Jun. 7, 2006

Related U.S. Application Data
(60) Provisional application No. 60/688,577, filed on Jun. 8, 2005.
(60) Provisional application No. 60/702,573, filed on Jul. 26, 2005.

(51) Int. Cl. A61K 31/56

(52) U.S. Cl. 514/169

(57) ABSTRACT

The invention relates to the field of pharmaceuticals for tumor-inhibitory effects. The 5-androstene 3β,17α diol (αAED) and 5-androstene 3β,17β diol (βAED), their esters and ethers, are taught herein to achieve tumor-inhibiting effect. The invention also relates to the field of pharmaceuticals for tumor-inhibitory effects and the use of 5-androstene 3β,7β,17β triol (βAET), 5-androstene 3β,7α,17β triol (αAET or 17α-AET) and their esters and ethers, are taught herein to achieve tumor-inhibiting effect.

### CROSS REFERENCE TO RELATED APPLICATIONS

This application claims priority to provisional application Ser. No. 60/688,577 filed on Jun. 8, 2005 and provisional application Ser. No. 60/702,573 filed on Jul. 26, 2005, both of which are incorporated herein in their entireties by reference thereto.

### BACKGROUND OF THE INVENTION

1. Field of the Invention

The invention relates to methods of treating gliomas in a subject by administering an effective amount of 5-androstenediol (AED), 5-androstenetriol (AET) or a derivative thereof to a subject.

2. Background of the Invention

The human central nervous system (CNS) is made up of two primary cell types: neurons and glia. Neurons are the main functional cell of the brain, while glia cells support the neuronal cells. Glial cells play a varied role in the CNS structure as disclosed in Miller, G., The Dark side of Glia, Science Vol. 308:778-7781 (2005) and Nimmerjahn et al., Resting Microglial Cells are Highly Dynamic Surveillants of Brain Parenchyma in Vitro, Science Vol. 308:1314-1318 (2005), which are hereby incorporated by reference in their entirety. Glia cells are commonly divided into several different subtypes including oligodendrocytes and astrocytes. Among other functions, oligodentrocytes cover the axons of neurons with sheaths of myelin. Astrocytes serve a variety of functions including the absorption of neurotransmitters and also function to create the blood-brain barrier.

Gliomas are tumors of glial cells, and more particularly, tumors of astroctytes and oligodentrocytes. Gliomas usually occur in the brain, which makes this type of cancer particularly dangerous.

Astrocytomas, or tumors derived from astrocytes or their precursors, are the most common type of glioma and include several subtypes including pilocytic astrocytoma, fibrillary astrocytoma, anaplastic astrocytoma and glioblastome multiforme. Glioblastome mutliforme (glioblastomas or GBM) is the most malignant of the gliomas. The World Health Organization has developed a grading system for astrocytomas that grades the severity of the astrocytoma on a scale from I to IV. The least aggressive gliomas are Grade I; the most aggressive and malignant gliomas are Grade IV. Glioblastome multiforme or GBM is classified as a Grade IV glioma.

Without therapy, patients diagnosed with GBM die within 3 months. The median mortality rate of GBM patients treated with conventional therapy is 12-18 months. The long-term survival rate of patients with GBM is 2-5% at five years.

Current therapies for treating GBM remain unsuccessful; mainstream treatments include surgery, radiation, and chemotherapy. As reported in Chang et al., Patterns of care for adults with newly diagnosed malignant glioma, JAMA, 293:557-564 (2005), incorporated herein by reference, the health care community has not provided standard treatment courses for individuals diagnosed with gliomas. Alternative treatments include the use of anti-angiogenesis agents (drugs that interfere with growth of blood vessels that feed the tumor), immunotoxins (a toxin is attached to an antibody that hones in on tumor cells), differentiating agents (which make the tumor behave in a less malignant way) and others.

GBM is a particularly infiltrative disease, with the tumor cells weaving in and out among normal brain and/or CNS structures. As disclosed in Fisher et al., Malignant Gliomas in 2005: Where to GO From Here?, JAMA 293: 615-617 (2005), incorporated herein by reference, surgical recession of the tumors remains difficult and often unsuccessful. Radiation therapy, which typically follows surgical recession, has also proven to be rarely curative or successful at preventing the progression of the disease. Similarly, chemotherapy remains ineffective. One of the obstacles encountered when treating GBM with chemotherapy is the blood brain barrier, which prevents chemotherapeutic agents from reaching the tumor site. While some agents and methods have been used to overcome this obstacle, current methods have not proven curative or particularly effective.

5-androstenediol (AED) is a naturally-occurring metabolite of dehydroepiandrosterone (DHEA), the most abundant product of the adrenal glands. AED may also arise from the metabolism of other steroids. AED exists in at least two epimeric forms: 5-androstene-3β-17α-diol (αAED) and 5-androstene-3β-17β-diol (βAED). 5-androstenetriol (AET) is also a naturally-occurring metabolite of dehydroepiandrosterone (DHEA), and AET can be generated from the metabolism of other steroids. AET exists in many epimeric forms, two of which are: 5-androstene-3β-7α-17β-triol (αAET) and 5-androstene-3β-7β-17β-triol (βAET).

DHEA, βAED, and βAET have in recent years been shown to increase and/or stimulate the immune response. As disclosed in U.S. Pat. No. 5,641,768 to Loria, DHEA, βAET, and βAET have been shown to up regulate the immune response and improve a host's response to infections. U.S. Pat. No. 5,641,768 is hereby incorporated fully by reference. Previous studies have also demonstrated that αAED inhibits the proliferation and induces apoptosis of the myeloid tumor cells, Raw 264.7, murine P388D1, and the human promyelocytic HL60 cells as reported in Huynh P N and Loria R M, J Leukoc Biol. Aug; 62(2):258-67 (1997), incorporated herein by reference. U.S. Pat. No. 5,912,240 (incorporated herein by reference) discloses the use of αAED to treat tumor growth. In contrast, no similar anti-proliferative affect by βAED or βAET has been reported.

Better treatments are needed to increase survival rates of subjects diagnosed with gliomas, and in particular Grade IV glioblastomas multifore or GBM. AED and derivatives thereof may thus hold promise for reducing side effects and mortality associated with gliomas. Similarly, AET and derivatives thereof may thus hold promise for reducing side effects and mortality associated with gliomas.

### SUMMARY OF THE INVENTION

The present invention provides a means of accelerating cell aging and programmed cell death in gliomas, and in particular glioblastomas. The practice of the invention involves the methods of treatment or administration of 5-androstene (hereinafter referred to in this application as AED) its epimeric forms 5-androstene 3β,17α diol (αAED or 17α-AED) and 5-androstene 3β,17β diol (βAED or 17β-AED), and esters and ethers thereof. The practice of the invention also involves the methods of treatment or administration of 5-androstene 3β,7β,17β triol (βAET or 17β-AET), and 5-androstene 3β,7α,17β triol (αAET or 17α-AET) and esters and ethers thereof.

### BRIEF DESCRIPTION OF THE DRAWINGS

FIG. 1 depicts the percent reduction in proliferation in rat F98 and T9 glioma cell lines and human U87 and T98 glioma cell lines in the presence of αAED or βAED.

FIG. 2 depicts the dose dependency of the antiproliferative effect of αAED or βAED in rat F98 glioma cells and human F98 glioma cells.

FIG. 3 depicts the morphological changes in rat F98 glioma cells cultured with AED and AET.

FIG. 4 depicts the antiproliferative effects and dose dependency of the epimers of AED and AET in human T98 glioma cells.

FIG. 5 depicts the antiproliferative effects and dose dependency of the epimers of AED and AET in rat F98 glioma cells.

### DETAILED DESCRIPTION OF THE INVENTION

The instant invention relates to the use of 5-androstene (AED), its epimeric forms 5-androstene 3β,17α diol (αAED or 17α-AED) and 5-androstene 3β,17β diol (βAED or 17β-AED), and their esters and ethers, to inhibit growth and accelerate cell aging, induce apoptosis and death of gliomas, and in particular glioblastomas. The structures of the α and β isomers of AED are given below: 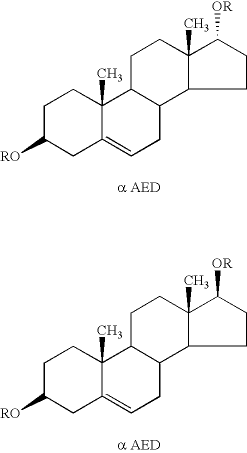
wherein R is a hydrogen. The invention also encompasses derivatives of AED wherein R may be H, alkenyl of 2-8 carbons, alkyl of 1-8 carbons, phenylalkyl of 1-4 carbons, phenyl or COR₂, wherein R₂ is H; alkyl of 1-8 carbons, alkenyl of 2-8 carbons, phenylalkyl wherein the alkyl has 1-4 carbons (including benzyl) or phenyl. Any phenyl moiety may have up to three substituents chosen from among hydroxy, carboxy of 1-4 carbons, halo, alkoxy of 1-4 carbons, alkyl of 1-4 carbons, or alkenyl of 2-4 carbons and wherein any alkyl may be a straight chain, branched chain, or the alkyl may be wholly or partially cyclized. The R groups need not be identical.

In addition to AED, the invention encompasses the use of derivatives of AED to treat gliomas in a subject. Derivatives of AED also include precursors, such as but not limited to dehydroepiandrosterone (DHEA) and AET, their metabolic intermediates thereof, and AED may also be substituted with protective groups which, on hydrolysis, yield AED. Hence, acylated and alkylated derivatives are useful as precursors to AED.

The instant invention also relates to the use of 5-androstene 3β,7β,17β triol (βAET or 17β-AET), and 5-androstene 3β,7α,17β triol (αAET or 17α-AET) and their esters and ethers, to inhibit growth and accelerate cell aging, induce apoptosis and death of gliomas, and in particular glioblastomas. The structure of the α and β isomer of AET is given below: 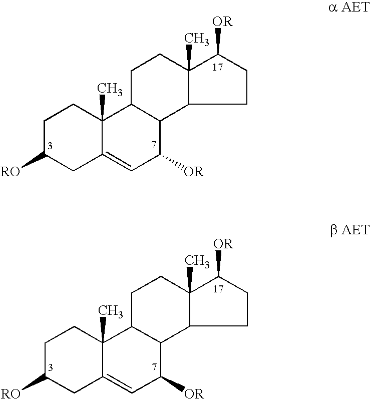
wherein R is a hydrogen. The invention also encompasses derivatives of AET wherein R may be H, alkenyl of 2-8 carbons, alkyl of 1-8 carbons, phenylalkyl of 1-4 carbons, phenyl or COR₂, wherein R₂ is H; alkyl of 1-8 carbons, alkenyl of 2-8 carbons, phenylalkyl wherein the alkyl has 1-4 carbons (including benzyl) or phenyl. Any phenyl moiety may have up to three substituents chosen from among hydroxy, carboxy of 1-4 carbons, halo, alkoxy of 1-4 carbons, alkyl of 1-4 carbons, or alkenyl of 2-4 carbons and wherein any alkyl may be a straight chain, branched chain, or the alkyl may be wholly or partially cyclized. The R groups need not be identical.

In addition to AET, the invention encompasses the use of derivatives of AET to treat gliomas in a subject. Derivatives of AET also include precursors, such as but not limited to dehydroepiandrosterone (DHEA) and AED, their metabolic intermediates thereof, and AET may also be substituted with protective groups which, on hydrolysis, yield AET. Hence, acylated and alkylated derivatives are useful as precursors to AET.

Other derivatives of AET, which may be used in the methods the present invention include, but are not limited to those compounds having the structure of formula 5, 6, 7, 8, 9, 10, 11, 12, 13 or 14

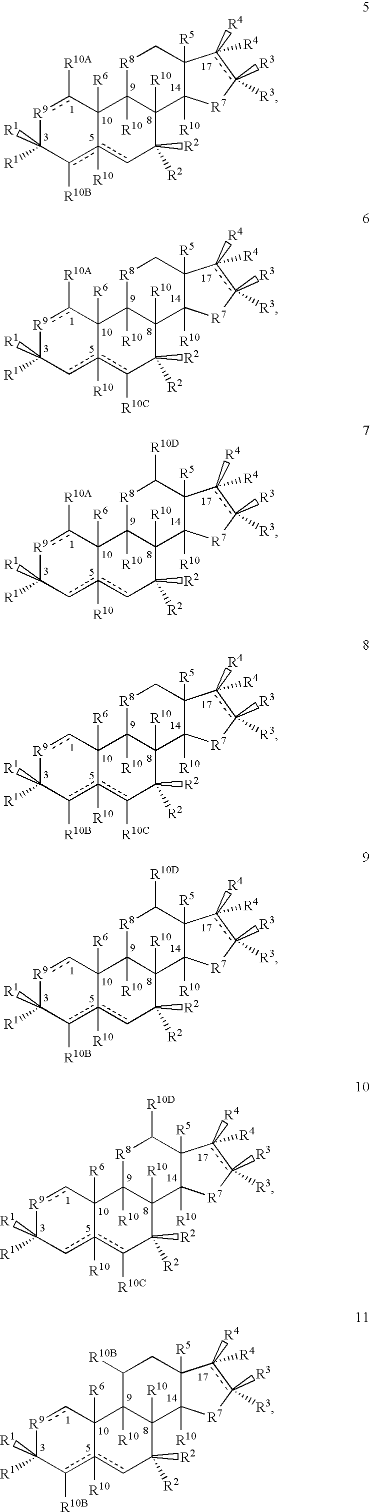

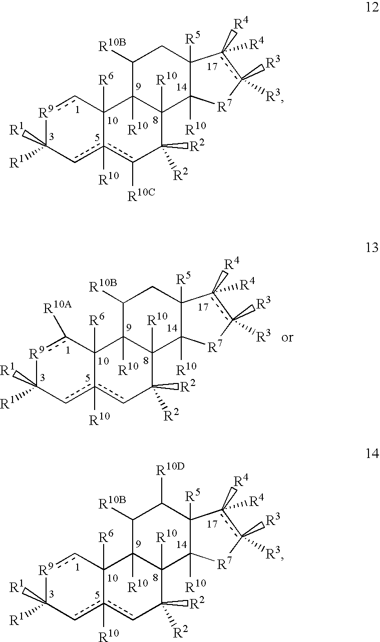

or a metabolic precursor or a metabolite thereof, wherein

R¹⁰ moieties at the 5 (if present), 8, 9 and 14 positions respectively are in the α,α,α,α; α,α,α,β; .α,α,β,α; α,β,α,α; β,α,α,α; α,α,β,β; .β,α,β,α; α,β,α,β; β,α,α,β; β,β,α,α; α,β,β,α; α,β,β,β; β,α,β,β; β,β,α,β; β,β,β,α; or β,β,β,β configurations,

wherein R^{10A}, R^{10B}, R^{10C}, R^{10D} and R^{10E} respectively are in the α,α; α,β; β, α or β,β configurations,

wherein, each R¹, R², R³, R⁴, R⁵, R⁶, R¹⁰, R^{10A}, R^{10B}, R^{10C}, R^{10D} and R^{10E} independently are —H, —OH, —OR^{PR}, SR^{PR}, —N(R^{PR})₂, —O—Si—(R¹³)₃, —CHO, —CHS, —CN, —SCN, —NO₂, —NH₂, —COOH, —OSO₃H, —OPO₃H, an ester, a thioester, a thionoester, a phosphoester, a phosphothioester, a phosphonoester, a phosphiniester, a sulfite ester, a sulfate ester, an amide, an amino acid, a peptide, an ether, a thioether, an acyl group, a thioacyl group, a carbonate, a carbamate, a halogen, an optionally substituted alkyl group, an optionally substituted alkenyl group, an optionally substituted alkynyl group, an optionally substituted aryl moiety, an optionally substituted heteroaryl moiety, an optionally substituted heterocycle, an optionally substituted monosaccharide, an optionally substituted oligosaccharide, a nucleoside, a nucleotide, an oligonucleotide, a polymer, or,

one more of R¹, R², R³, R⁴, R⁵, R⁶, R¹⁰, R^{10A}, R^{10B}, R^{10C}, R^{10D} and R^{10E} are ═O, ═S, ═N—OH, ═CH₂, ═CH—CH₃, or an independently selected spiro ring and the hydrogen atom or the second variable group that is bonded to the same carbon atom is absent, or,

one or more of two adjacent R¹-R⁶, R¹⁰, R^{10A}, R^{10B}, R^{10C}, R^{10D} and R^{10E} comprise an independently selected epoxide, acetal, a thioacetal, ketal or thioketal;

R⁷ is —C(R¹¹)₂—, —C(R¹¹)₂—C(R¹¹)₂—, —C(R¹¹)₂—C(R¹⁰)₂—C(R¹¹)₂—, —C(R¹¹)₂—O—C(R¹⁰)₂—, —C(R¹⁰)₂—S—C(R¹⁰)₂—, —C(R¹⁰)₂—NR^{PR}—C(R¹⁰)₂—, —O—, —O—C(R¹⁰)₂—, —S—, —S—C(R¹⁰)₂—, NR^{PR} or —NR^{PR}—C(R¹⁰)₂—;

R⁸ and R⁹ independently are —C(R¹⁰)₂—, —C(R¹⁰)₂—C(R¹⁰)₂—, —O—, —O—C(R¹¹)₂—, —S—, —S—C(R¹⁰)₂—, —NR^{PR}— or —NR^{PR}—C(R¹⁰)₂—, or one or both of R⁸ or R⁹ independently are absent, leaving a 5-membered ring;

R¹³ independently is C₁₋₆ alkyl; and

R^{PR} independently is —H or a protecting group. In typical embodiments, one or two of R^{10A}, R^{10B}, R^{10C}, R^{10D} and R^{10E} are not hydrogen or one R⁴ is —NH₂, an opotionally substituted amine, —N(R^{PR})², ═NOH, ═NO-optionally substituted alkyl, an amide or an N-linked amino acid.

Other embodiments include (1) certain new formula 1 compounds, which are new chemical entities, (2) compositions that comprise a formula 1 compound and another compound or an excipient, (3) formulations that comprise a formula 1 compound and 1, 2, 3, 4, 5, 6 or more excipients. The formulations can be designed for human pharmaceutical use or they can be suitable for veterinary use. Therapeutic use embodiments include (1) use of a formula 1 compound for the preparation of a medicament and (2) use of a formula 1 compound for the preparation of a medicament for the prophylaxis or treatment of a condition or symptom disclosed herein. Specific compounds for use in the methods of the present invention include, but are not limited to those compounds listed in U.S. Ser. No. 10/728,400, filed Dec. 5, 2003, which is a continuation-in-part of U.S. Ser. No. 10/651,515, filed Aug. 28, 2003, both of which are incorporated by reference in their entirety.

As used herein, the term “treatment” is used to indicate a procedure which is designed ameliorate one or more causes, symptoms, or untoward effects of an abnormal condition in a subject. Likewise, the term “treat” is used to indicate performing a treatment. The treatment can, but need not, cure the subject, i.e., remove the cause(s), or remove entirely the symptom(s) and/or untoward effect(s) of the abnormal condition in the subject. More particularly, the term “treatment” is also used to indicate a procedure which is designed to inhibit growth and accelerate cell aging, induce apoptosis and death of gliomas. Additionally, “treatment” means the application of the methods of the present invention to reduce, stall, or inhibit the growth of or proliferation of tumor cells, including but not limited to gliomas, and more particularly glioblastomas.

As used herein, the term “subject” is used interchangeably with the term “patient,” and is used to mean an animal, in particular a mammal, and even more particularly a non-human or human primate

As used herein, the term “administer” and “administering” are used to mean introducing at least one compound into a subject. When administration is for the purpose of treatment, the substance is provided at, or after the diagnosis of abnormal cell growth, and more particularly at, or after the diagnosis of gliomas. The therapeutic administration of this substance serves to inhibit cell growth of the tumor or abnormal cell growth. The route of administration of the compound includes, but is not limited to, topical, transdermal, intranasal, vaginal, rectal, oral, subcutaneous intravenous, intraarterial, intracranial, intramuscular, intraosseous, intraperitoneal, epidural and intrathecal.

As used herein, the term “coadminister” is used to mean that each of at least two compounds be administered during a time frame wherein the respective periods of biological activity overlap. Thus the term includes sequential as well as coextensive administration of the compounds of the present invention. If more than one substance is coadministered, the routes of administration of the two or more substances need not be the same. The scope of the invention is not limited by the identity of the substance which may be coadministered. For example, αAED, βAED, αAET or βAET may be coadministered with an AED or AET derivative or other pharmaceutically active substances, such as vinca alkaloids, nucleic acid inhibitors, platinum agents, interleukin-2, interferons, alkylating agents, antimetabolites, corticosteroids, DNA intercalating agents, anthracyclines, and ureas. Examples of specific agents in addition to those exemplified herein, include hydroxyurea, 5-fluorouracil, anthramycin, asparaginase, bleomycin, dactinomycin, dacabazine, cytarabine, busulfan, thiotepa, lomustine, mechlorehamine, cyclophosphamide, melphalan, mechlorethamine, chlorambucil, carmustine, 6-thioguanine, methotrexate, etc.

As used herein, the term an “effective amount” is used to mean an amount of a substance that can elicit a desired response without excessive side effects. The response to the pharmaceutically effective amount may be a cellular, organ or tissue-specific response, or system response.

As used herein, the term “prevent,” as it relates to tumors or abnormal cell growth, indicates that a substance of the present invention is administered to a subject to at least partially inhibit the growth, division, spread, or proliferation of tumor cells, and more particularly gliomas and glioblastomas. Of course, the term “prevent” also encompasses prohibiting entirely tumors, particularly glioblastomas, or any of its associated symptoms, from detectably appearing. Thus a subject may be “pretreated,” by using the substances of the present invention to prevent tumors, particularly gliobalstomas, from arising. The phrase “preventing the progression,” as it relates to tumors, is used to mean a procedure designed to at least partially inhibiting the detectable appearance of one or more additional tumors in a patient already exhibiting one or more symptoms of the presence of a tumor, and is also used to mean at least partially prohibiting the already-present symptoms of cancer from worsening in the subject.

A medicament comprising a substance of the present invention, for example, αAED, βAED, αAET, βAET or a derivative thereof, may be prepared by standard pharmaceutical techniques known in the art, depending upon the mode of administration and the particular disease to be treated. The medicament will usually be supplied as part of a sterile, pharmaceutical composition which will normally include a pharmaceutically acceptable carrier. This pharmaceutical composition may be in any suitable form, (depending upon the desired method of administering it to a subject). It may be provided in unit dosage form, will generally be provided in a sealed container and may be provided as part of a kit, which may include instructions for use and/or a plurality of unit dosage forms.

The pharmaceutical composition may be adapted for administration by any appropriate route, for example by the topical, transdermal, intranasal, vaginal, rectal, oral, subcutaneous intravenous, intraarterial, intracranial, intramuscular, intraosseous, intraperitoneal, epidural and intrathecal route. Such compositions may be prepared by any method known in the art of pharmacy, for example by admixing the active ingredient with the carrier(s) or excipient(s) under sterile conditions.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions). Suitable excipients for tablets or hard gelatine capsules include lactose, maize starch or derivatives thereof, stearic acid or salts thereof. Suitable excipients for use with soft gelatine capsules include for example vegetable oils, waxes, fats, semi-solid, or liquid polyols etc. For the preparation of solutions and syrups, excipients which may be used include for example water, polyols and sugars. For the preparation of suspensions oils (e.g. vegetable oils) may be used to provide oil-in-water or water in oil suspensions. In certain situations, delayed release preparations may be advantageous and compositions which can deliver, for example, αAED, βAED, αAET, βAET or a derivative thereof in a delayed or controlled release manner may also be prepared. Prolonged gastric residence brings with it the problem of degradation by the enzymes present in the stomach and so enteric-coated capsules may also be prepared by standard techniques in the art where the active substance for release lower down in the gastro-intestinal tract.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6):318 (1986).

Pharmaceutical compositions adapted for topical administration may be formulated as ointments, creams, suspensions, lotions, powders, solutions, pastes, gels, sprays, aerosols or oils. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration wherein the carrier is a solid include a coarse powder having a particle size for example in the range 20 to 500 microns which is administered in the manner in which snuff is taken, i.e., by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurised aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water, alcohols, polyols, glycerine and vegetable oils, for example. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets. The pharmaceutical compositions may contain preserving agents, solubilising agents, stabilising agents, wetting agents, emulsifiers, sweeteners, colourants, odourants, salts (substances of the present invention may themselves be provided in the form of a pharmaceutically acceptable salt), buffers, coating agents or antioxidants. They may also contain therapeutically active agents in addition to the substance of the present invention.

Dosages of the substance of the present invention can vary between wide limits, depending upon the location and severity of the glioma, the age and condition of the individual to be treated, etc. and a physician will ultimately determine appropriate dosages to be used.

Materials and Methods

αAED, βAED, αAET, and βAET, are commercially available. Unless otherwise indicated, all steroids were dissolved in DMSO:ETHOL (1:1 v/v). Stock solutions were refrigerated and brought to room temperature before use. For testing, stock solutions were diluted in media immediately before use. The final concentration of vehicle (PEG400/ETOH, 50%/50%) was always less than 0.01% by volume in all samples, and this concentration had no significant cytotoxic effect on the rat T9 and F98 glioma cell lines and the human U87 and T98 glioma cell lines.

The rat F98 cell line was obtained from the American Type Culture Collection (ATCC No. CRL-2397). These cells are tumorgenic and have biological characteristics that closely resemble those of human glioblastoma. The F98 cells are weakly immunogenic. The rat T9 cell line was originally induced by the repeated intravenous injection of N-nitrosemoethylurea in a Fischer F344 rat. The T9 cell line is tumorgenic and immunogenic. The human U-87 cell line was obtained from American Type Culture Collection (ATCC No. HTB-14). These cells are tumorgenic and are derived from malignant gliomas. The human T98 cell line was obtained from the American Type Culture Collection (ATCC No. CRL-1690). These cells are derived from the brain of a subject diagnosed with glioblastoma multiforme.

Cell Growth Testing and Proliferation

Cells were grown in Complete Media (CM) consisting of Dulbecco's Modified Eagle Medium. Cells were initially seeded at a density of 1×10⁴ cells per 0.2 ml in 96 well plates and grown at 37° C. During the growth phase, the media was not changed. Proliferation was measured by tritium uptake (as a measure of DNA synthesis). One microcurie (1 μC) was added to the cells and incubated overnight. The 96 well plate was then refrigerated and counted in a topcount cell plate harvester.

The following examples are meant to be illustrative and not intended to limit the scope of the invention described herein.

### EXAMPLES

### Example 1

### Inhibition of Proliferation of Human and Rodent Glioma Cells

Rat F98 and T9 glioma cells and Human F98 and U87 glioma cells were cultured in 96 well plates at a density of approximately 1×10⁴ cells per well. Cells were treated with 500 μm of αAED or βAED or vehicle and cultured for 3 days. The cultures were pulsed with 3H-TdR for the last 15 hours. The incorporation of 3H-TdR was used to measure cellular proliferation. At the end of 3 days, the proliferation of the cells were measured as described above.

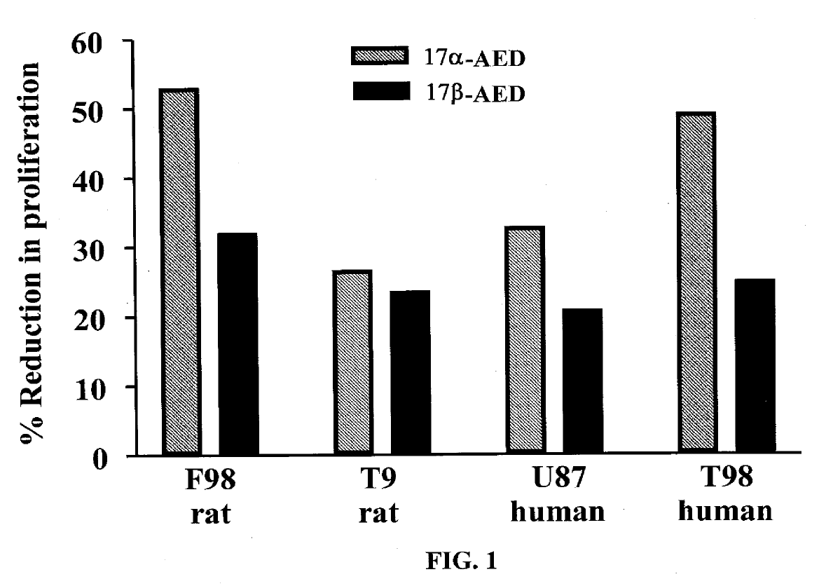

As seen in FIG. 1, AED had a significant negative effect on the proliferation of the glioma cell lines. More particularly, αAED inhibited cell growth of rat F98 glioma cells, rat T9 glioma cells, human U87 glioma cells, and human T98 glioma cells when compared to controls. Similarly, βAED inhibited cell growth of the rat F98 glioma cells, rat T9 cells, human U87 glioma cells, and human T98 glioma cells. This data demonstrates that both epimers of AED have a significant antiproliferative effect on glioma cells. Previous to this finding, there have been no indications that βAED inhibited proliferation of tumor cell lines, and in particular glioma cell lines.

### Example 2

### Suppression of Glioma Growth is Dose Dependent

Rat F98 glioma cells and human F98 glioma cells were grown and cultured as in example 1 above. The cells were cultured in the presence of αAED or βAED in titrated concentrations of 500 μm, 50 μm, 5.0 μm, and 0.5 μm. After 3 days, glioma cell proliferation was measured as described above.

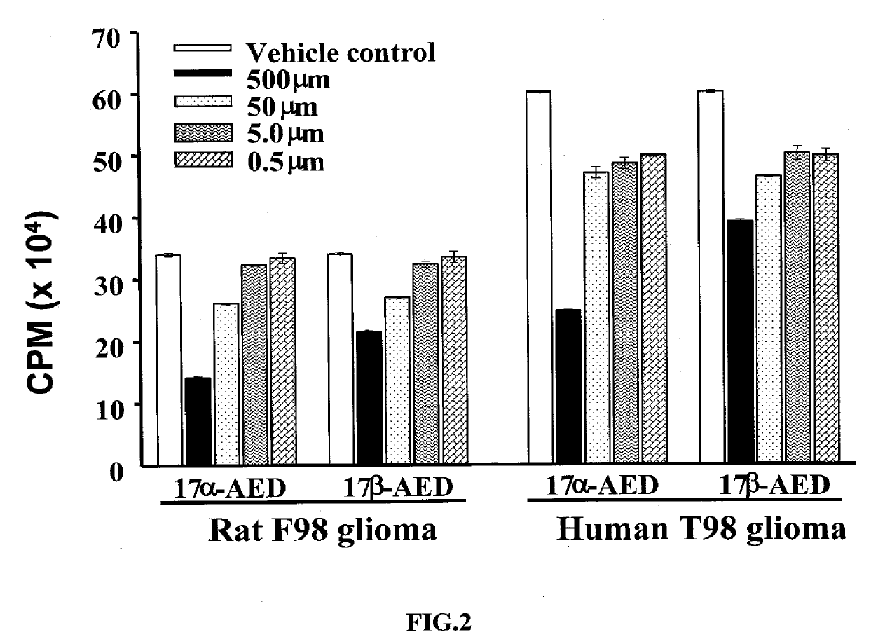

As seen if FIG. 2, αAED and βAED show significant antiproliferative effects on the rat F98 glioma cells and human T98 glioma cells. Furthermore, as seen in FIG. 2, the antiproliferative effect is dose dependent. At 500 μm concentrations, αAED and βAED inhibited the proliferation of rat F98 glioma cells and human T98 glioma cells. As can be seen in FIG. 2, as the concentrations of αAED and βAED decreased, so does the corresponding antiproliferative effect. These results show that growth inhibition by AED is dose dependent.

### Example 3

### Morphological Data Showing Effects of Androsterene on Rat F98 Gliomas

Cells were cultured as described in example 1 for 24 hours. Cells were cultured with 500 mm of αAED, βAED, βAET, and vehicle. After culture, the cells were viewed in situ under 20× magnification using a reverse image microscope.

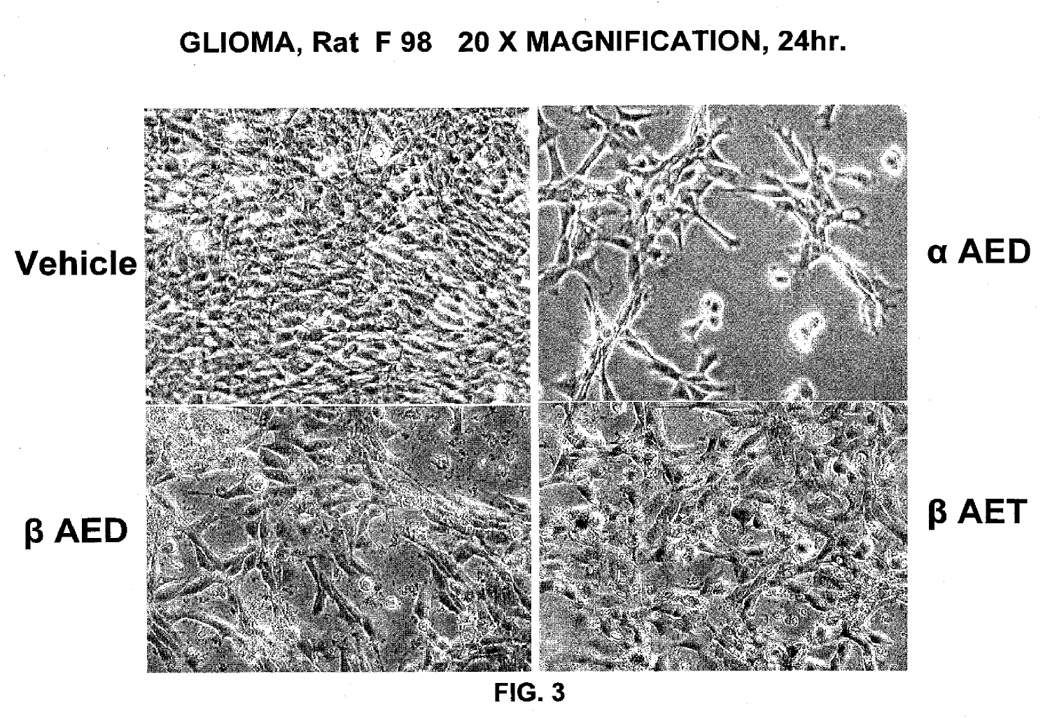

As can be seen in FIG. 3, significant morphological changes occurred in the rat F98 glioma cells cultured with αAED, βAED, or βAET as compared to vehicle treated cells. The results indicate that αAED exhibits the most drastic morphological changes as compared to the cells treated with vehicle. As demonstrated in FIG. 3, the vehicle treated cells appear normal, whereas the αAED, βAED, and βAET treated cells display morphology of apoptasis including cell shrinkage, rounding, extensive vascuolization, partial detachment and further demonstrate the lobulated appearance of apoptotic cells. FIG. 3 shows that βAED and βAET produce significant morphological changes indicative of antiproliferative effects on the glioma cells, a fact heretofore unreported.

### Example 4

### Inhibition of Proliferation and Dose Dependency of Androsterene on Human Glioma Cells

Human T98 glioma cells were cultured in 96 well plates at a density of approximately 1×10⁴ cells per well. The cells were cultured in the presence of αAED, βAED, αAET, βAET, or vehicle in titrated concentrations of 400 μm, 200 μm, 100 μm, and 50 μm. The cultures were pulsed with 3H-TdR for the last 15 hours. The incorporation of 3H-TdR was used to measure cellular proliferation. At the end of 3 days, the proliferation of the cells were measured as described above.

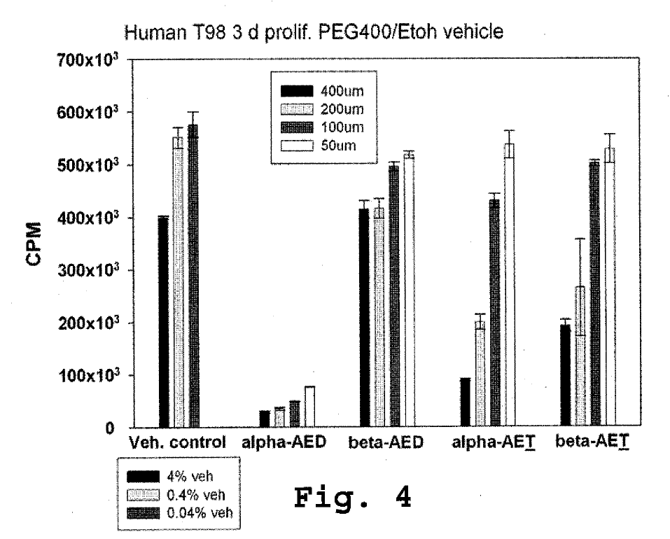

As seen in FIG. 4, AED and AET had significant negative effects on the proliferation of the glioma cell lines. More particularly, αAED significantly inhibited cell growth of human T98 glioma when compared to controls. Similarly, βAED inhibited cell growth of the human T98 glioma cells. Both αAET and βAET displayed significant effects on the proliferation of human T98 glioma cell lines. This data demonstrates that both epimers of AED and both epimers of AET have a significant antiproliferative effect on human T98 glioma cells. Furthermore, FIG. 4 shows the dose dependent effects of the epimers of AED and AET at increasing concentrations of AED and AET. Previous to this finding, there have been no indications that βAED or βAET inhibited proliferation of tumor cell lines, and in particular glioma cell lines.

### Example 5

### Inhibition of Proliferation and Dose Dependency of Androsterene on Rat Glioma Cells

Rat F98 glioma cells were cultured in 96 well plates at a density of approximately 1×10⁴ cells per well. The cells were cultured in the presence of αAED, βAED, αAET, βAET, or vehicle in titrated concentrations of 400 μm, 200 μm, 100 μm, and 50 μm. The cultures were pulsed with 3H-TdR for the last 15 hours. The incorporation of 3H-TdR was used to measure cellular proliferation. At the end of 3 days, the proliferation of the cells were measured as described above.

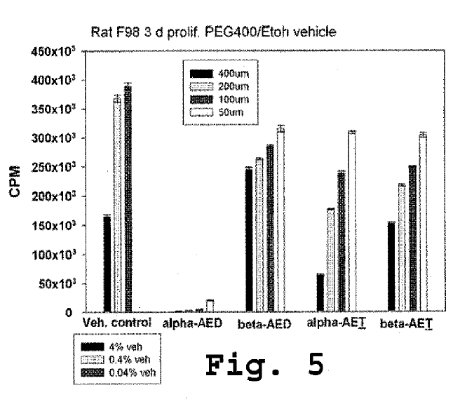

As seen in FIG. 5, AED and AET had significant negative effects on the proliferation of the glioma cell lines. More particularly, αAED significantly inhibited cell growth of rat F98 glioma when compared to controls. Similarly, βAED inhibited cell growth of the rat F98 glioma cells. Both αAET and βAET displayed significant effects on the proliferation of rat F98 glioma cell lines. This data demonstrates that both epimers of AED and both epimers of AET have significant antiproliferative effects on rat F98 glioma cells. Furthermore, FIG. 5 shows the dose dependent effects of the epimers of AED and ALT at increasing concentrations of AED and AET. Previous to this finding, there have been no indications that βAED or βAET inhibited proliferation of tumor cell lines, and in particular glioma cell lines.

While the invention has been described and illustrated herein by references to various specific material, procedures and examples, it is understood that the invention is not restricted to the particular material, combinations of material, and procedures selected for that purpose. Numerous variations of such details can be implied and will be appreciated by those skilled in the art.

What is claimed is:

1. A method of inhibiting glioma cell proliferation comprising administration of a tumor proliferation inhibiting effective amount of at least one tumor-inhibiting agent which is βAED or an ester or ether thereof of the formula: 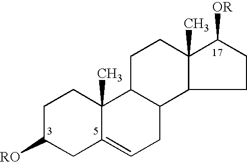
wherein R₁ may be H, alkenyl of 2-8 carbons, alkyl of 1-8 carbons, phenylalkyl of 1-4 carbons, phenyl or COR₂, wherein R₂ is H; alkyl of 1-8 carbons, alkenyl of 2-8 carbons, phenylalkyl wherein the alkyl has 1-4 carbons (including benzyl) or phenyl. Any phenyl moiety may have up to three substituents chosen from among hydroxy, carboxy of 1-4 carbons, halo, alkoxy of 1-4 carbons, alkyl of 1-4 carbons, or alkenyl of 2-4 carbons and wherein any alkyl may be a straight chain, branched chain, or the alkyl may be wholly or partially cyclized.

2. The method of claim 2, wherein the glioma is multiforme glioblastoma.

3. The method of claim 1, wherein the tumor-inhibiting agent is administered orally.

4. The method of claim 1, wherein the tumor-inhibiting agent is administered parenterally.

5. The method of claim 1, wherein the tumor-inhibiting agent is applied to mucosal tissue.

6. The method of claim 1, wherein the tumor-inhibiting agent is applied as a spray or mist.

7. The method of claim 1, wherein the tumor-inhibiting agent is applied to the site of the tumor or tumor bed.

8. The method of claim 1, wherein the tumor-inhibiting agent is administered as a patch.

9. The method of claim 1, wherein the tumor-inhibiting agent is coadministered with derivatives of AED.

10. The method of claim 1, wherein the tumor-inhibiting agent is coadministered with another pharmaceutically active substances selected from the group consisting of vinca alkaloids, nucleic acid inhibitors, platinum agents, interleukin-2, interferons, alkylating agents, antimetabolites, corticosteroids, DNA intercalating agents, anthracyclines, and ureas.

11. The method of claim 1, wherein the tumor-inhibiting agent is coadministered with another agent selected from the group consisting of hydroxyurea, 5-fluorouracil, anthramycin, asparaginase, bleomycin, dactinomycin, dacabazine, cytarabine, busulfan, thiotepa, lomustine, mechlorehamine, cyclophosphamide, melphalan, mechlorethamine, chlorambucil, carmustine, 6-thioguanine and methotrexate.
